(19) United States
(12) Patent Application Publication
(10) Pub. No.: US 2024/0225915 A1
(43) Pub. Date: Jul. 11, 2024

(54) COMPOSITIONS FOR FIGHTING MALODORS

(71) Applicant: SYMRISE AG, Holzminden (DE)

(72) Inventors: Edison DIAZ, Goslar (DE); Marco SINGER, Holzminden (DE); Sven SIEGEL, Höxter (DE)

(21) Appl. No.: 18/566,823

(22) PCT Filed: Jun. 10, 2021
(86) PCT No.: PCT/EP2021/065677

§ 371 Date: Dec. 4, 2023

(51) Int. Cl. A61F 13/49; A61F 13/15; A61F 13/472; A61F 13/51; A61F 13/53

(52) U.S. Cl. CPC A61F 13/49; A61F 13/472; A61F 2013/1556; A61F 2013/51076; A61F 2013/530693

(57) ABSTRACT

Suggested is a composition for fighting malodors comprising or consisting of (a) at least one physiological cooling gent and (b) at least one water-absorbing polymer and/or hydrogel.

The present invention refers to the area of malodor reduction and concerns a new composition for fighting particular female body odors, a method for fighting and a method for determining such malodors.

### BACKGROUND OF THE INVENTION

Body odor is primarily the term used to describe all odoriferous body exhalations from people via the skin. The most clearly perceptible odor is that of sweat, whereby only the secretions of the apocrine sweat glands, which are located primarily in the armpits, are odorous. However, the odorants are only produced during the bacterial decomposition of the substances contained in underarm perspiration, including the body's own fats and proteins. For many people, unpleasant body odors represent a serious problem. The market for deodorants is currently worth around EUR 20 billion worldwide, and is expected to increase by 10% by 2025.

However, combating odors associated with the excretion of urine and menstrual blood in particular remains a particular challenge. Corresponding hygiene products, such as diapers, panty liners and sanitary napkins, contain so-called superabsorbers to absorb body fluids. These superabsorbers have the property of absorbing liquids, whereby the substances expand many times their volume. Since the liquids are stored within the polymer matrix, the superabsorbers remain dry on the outside.

Even when the liquids are absorbed, however, the unpleasant odors remain. Usually, hygiene products are therefore elaborately perfumed, but this serves the sole purpose of masking the bad odors. This is sometimes more or less successful, but in particular it happens time and again that the odor components not only mix, but even intensify unpleasantly.

### Relevant Prior Art

U.S. Pat. No. 6,379,652 B1 (COLGATE) discloses a method to suppress oral malodour and provide long lasting breath protection wherein there is applied to the oral cavity of the user an oral composition comprising an orally acceptable vehicle containing therein a flavor system comprised of a mixture of an essential oil and a coolant compound which is a menthyl ester of naturally occurring hydrocarboxylic acids having 2 to 6 carbon atoms esterified with C1-C4 alkyl groups.

US 2014 377207 AA and US 2014 378920 AA (P&G) refer to absorbent articles defined by their thiol vapor pressure comprising one or more complexed or encapsulated compounds compounds selected from: melonal, adoxal, trans-2-hexenal, ligustral, Floral Super, Florhydral, 5-methyl-2-thiophene-carboxaldehyde, hydratropic aldehyde, undecenal, 9-undecenal, 10-undecenal, trans-4-decenal, cis-6-nonenal, isocyclocitral, precyclemone b, (E)-2,(z)-6-nonadienal, undecyl aldehyde, methyl-octyl-acetaldehyde, Lauric aldehyde, silvial, vanillin, floralozone are particularly effective in reducing malodours coming from degradation of proteinaceous materials such as food, menses or feces.

EP 1239890 B1 (SYMRISE) covers a method of reducing the perception of male or female body odour, comprising administering a deodorant composition comprising a cross-adapting agent in an amount effective to reduce perception of body odour, wherein the cross-adapting agent is selected from the group consisting of: 1-(5,5-dimethyl-1-cyclohexen-1-yl)4-penten-1-one; hexyl salicylate; 2,2-dimethyl-3-(methylphenyl)propanol; menthyl acetate rf; 1-(4-isopropylcyclohexyl)ethanol; 2,4-dimethyl-3-cyclohexene-1-carboxyaldehyde, and combinations thereof.

EP 1474095 B1 (SYMRISE) claims the use of an ester selected from the group consisting of: isomenthyl acetate; isomenthyl propionate; isomenthyl isobutyrate; isomenthyl crotonate; isomenthyl butyrate as odor neutralizer.

### OBJECT OF THE INVENTION

Therefore, the task of the present invention has been providing new alternative compositions for sanitary articles that do not overpower bad odors; in particular an odor associated with female body odors, urine, ammonia und protein degradation products, by the stronger inherent odor, but neutralizes them already at low concentrations.

### BRIEF DESCRIPTION OF THE INVENTION

A first object of the present invention refers to a composition for fighting malodors comprising or consisting of
(a) at least one physiological cooling agent and
(b) at least one water-absorbing polymer and/or hydrogel.

Although malodor counteraction is a well investigated matter; nevertheless the interaction between menthyl acetate representing a typical a fragrance ingredient and water-absorbent polymer commonly used in baby wipes, feminine hygiene wipes or similar applications is a new and surprising way for releasing proper high malodor counteraction.

### Physiological Cooling Agents

The physiological cooling agents forming component (a) are preferably selected from the group formed by the species depicted in the following table (including their optical isomers and racemates):

| Component | FEMA/GRAS | Trademark | Supplier |
|---|---|---|---|
| Menthol | | | |
| Menthol glyceryl acetal | 3807 | Frescolat° MGA | Symrise AG |
| Menthol glyceryl ketal | 3808 | Frescolat° MGA | Symrise AG |
| Menthol menthyl ether | | | |
| Menthone glyceryl acetal | | | |
| Menthone glyceryl ketal | | | |
| Menthoxy-1,2-propandiol | 3784 | | |
| Menthoxy-2-methyl-1,2-propanediol | 3849 | | |
| Menthyl acetate | | SymFresh° RF | Symrise AG |
| Menthyl ethylene glycol carbonate | 3805 | Frescolat° MGC | Symrise AG |
| Menthyl formiate | | | |
| Menthyl glutamate | 4006 | | |
| Menthyl glycerol carbonate | | | |
| Menthyl hydroxy isobutyrate | | | |
| Menthyl isobutyrate | | | |
| Menthyl lactate | | Frescolat° ML | Symrise AG |
| Menthyl malonate | | | |
| Menthyl methyl ether | | | |
| Menthyl N-ethyl oxamate | | | |
| Menthyl propylene glycol carbonate | 3806 | Frescolat° MPC | Symrise AG |
| Menthyl pyroglutamate | | | |
| Menthyl-(2-methoxy)acetate | | | |
| Menthyl-(2-methoxyethoxy)acetate | | | |
| Menthyl succinate | 3810 | | |
| O-Menthyl succinic acid ester amide | | | |
| O-Menthyl succinic acid ester-NN-(dimethyl)amide | | | |
| Menthane carboxylic acid-N-(4-cyanophenyl)amide | | | |
| Menthane carboxylic acid-N-(4-cyanomethyl- | | | |
| phenyl)amide | | | |
| Menthane carboxylic acid-N-ethylamide (WS-3) | | | |
| (WS-4) | | | |
| N^{α}-(menthane carbonyl) glycinc ethylester (WS-5) | | | |
| (1R,2S,5R)-N-(4-Methoxyphenyl)-5-methyl-2-(1- | | | |
| isopropyl)cyclohexane-carboxamide (WS-12) | | | |
| (WS-14) | | | |
| 2,3-dimethyl-2-(2-propyl)-butyric acid-N- | | | |
| methylamide (WS23) | | | |
| Isopulegol acetate | | | |
| p-Menthane-3,8-diol | | | |
| Cubebol | | | |
| 3-Methyl-2(1-pyrrolidinyl)-2-cyclopentene-1-one) | | | |
| Tetrahydropyrimidine-2-one | | | |
| N-(2-(pyridin-2-yl)ethyl)-3-p-menthane- | | | |
| carboxamide | | | |
| [(1R,2S,5R)-2-isopropyl-5-methyl-cyclohexyl] 2- | | X Cool | Symrise AG |
| (ethylamino)-2-oxo-acetate | | | |

A first important representative of the substances forming component (b) is monomenthyl succinate, which as a substance was patented as early as 1963 by Brown & Williamson Tobacco Corp. (U.S. Pat. No. 3,111,127) and as a refrigerant is the subject of property rights U.S. Pat. Nos. 5,725,865 and 5,843,466 (V.Mane Fils). Both the succinate and the analogous monomenthyl glutarate are important representatives of monomenthyl esters based on di- and polycarboxylic acids:

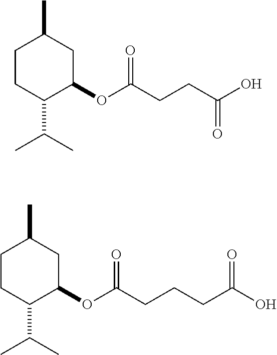 [SMILES: CC(C)[C@@H]1CC[C@@H](C)C[C@H]1OC(=O)CCC(=O)O ; CC(C)[C@@H]1CC[C@@H](C)C[C@H]1OC(=O)CCCC(=O)O]

Examples of applications of these substances can be found, for example, in the printed documents WO 2003 043431 (Unilever) or EP 1332772 A1 (IFF).

The next important group of menthol compounds preferred in the sense of the invention comprises carbonate esters of menthol and polyols, such as glycols, glycerol or carbohydrates, such as menthol ethylene glycol carbonates, menthol propylene glycol carbonates, menthol 2-methyl-1,2-propanediol carbonates or the corresponding sugar derivatives:

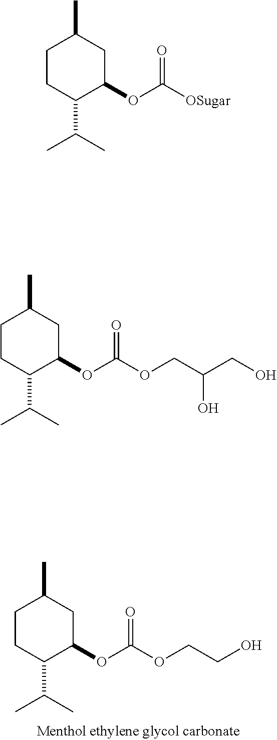

The use of such substances as a cooling agent for cigarettes is, for example, the subject of the 1968 publication U.S. Pat. No. 3,419,543 (Mold et al.); their use as a physiological cooling agent is claimed in DE 4226043 A1 (H&R).

In the sense of the invention, the menthol compounds menthyl lactate) and, in particular, menthone glyceryl acetal or menthone glyceryl ketal are preferred.

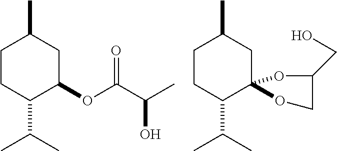

The former structure is obtained by esterification of lactic acid with menthol, the latter by acetylation of menthone with glycerol (cf. DE 2608226 A1, H&R). This group of compounds also includes 3-(I-menthoxy)-1,2,propanediol, also known as Cooling Agent 10 (U.S. Pat. No. 6,328,982, TIC), and 3-(I-menthoxy)-2-methyl-1,2,propanediol, which has an additional methyl group.

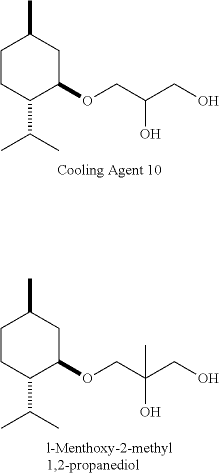 [SMILES: CC(C)[C@@H]1CC[C@@H](C)C[C@H]1OCC(O)CO ; CC(C)[C@@H]1CC[C@@H](C)C[C@H]1OCC(C)(O)CO]

For example, 3-(I-menthoxy)-1,2,propanediol is prepared starting from menthol according to the following scheme (see U.S. Pat. No. 4,459,425, Takasago):

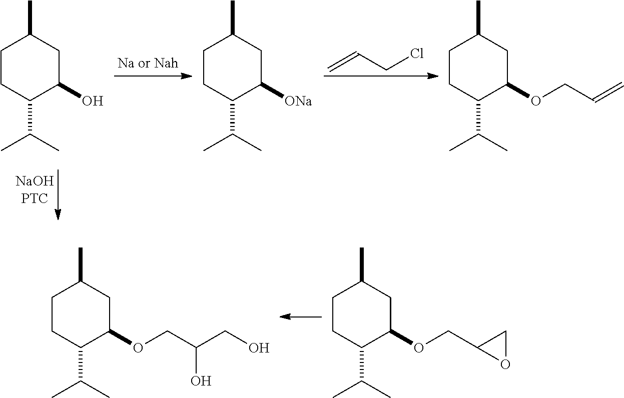

Alternative routes in which menthol is reacted with epichlorohydrin in the first step are described in U.S. Pat. Nos. 6,407,293 and 6,515,188 (Takasago). The following is an overview of preferred menthol compounds characterized by CO bonding:

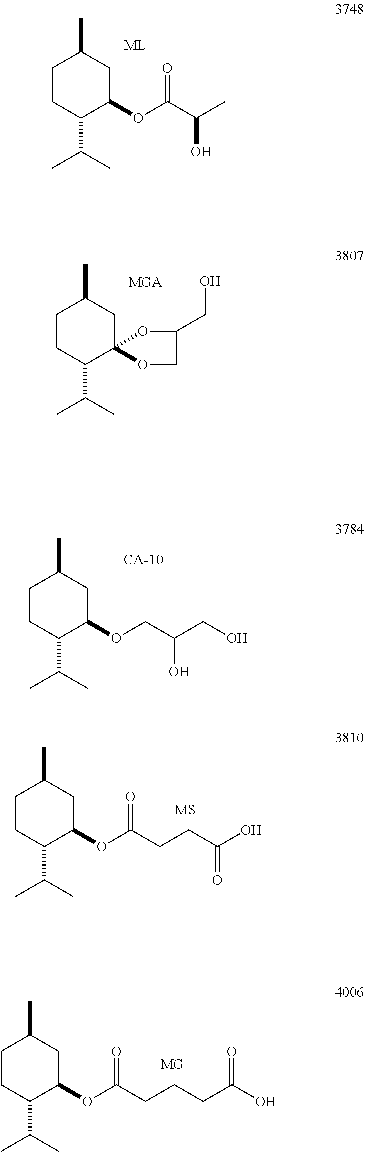

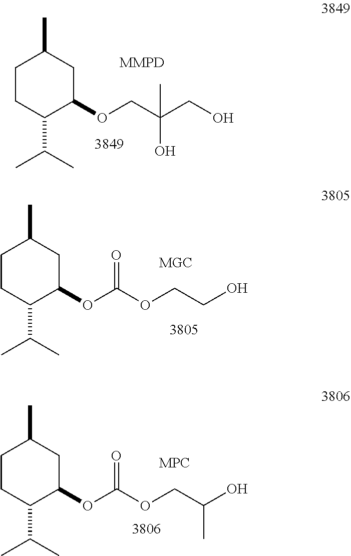

Among these substances, menthone glyceryl acetal/ketal and menthyl lactate as well as menthol ethylene glycol carbonate and menthol propylene glycol carbonate.

In the 1970s, menthol compounds were developed for the first time which have a C-C bond in the 3-position and of which a number of representatives can also be used in the sense of the invention. These substances are generally referred to as WS types. The basic body is a menthol derivative in which the hydroxyl group is replaced by a carboxyl group (WS-1). All other WS types are derived from this structure, such as the species WS-3, WS-4, WS-5, WS-12, WS-14 and WS-30, which are also preferred in the sense of the invention. The following two diagrams show the synthesis routes:

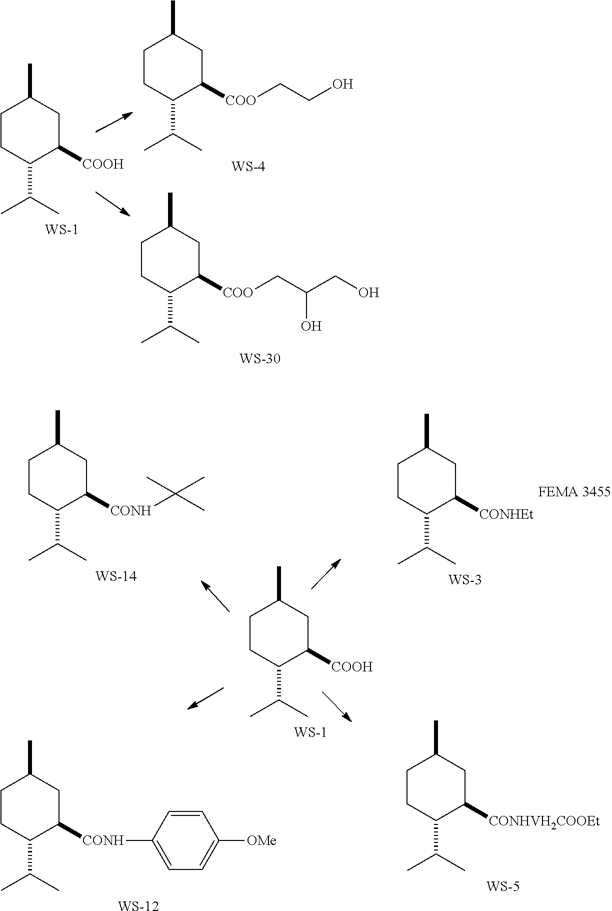

The esters derived from WS-1 are described, for example, in U.S. Pat. No. 4,157,384, and the corresponding N-substituted amides in J. Soc. Cosmet. Chem. pp. 185-200 (1978).

The preferred agents are selected from the group consisting of:
Menthol glyceryl acetal
Menthol glyceryl ketal
Menthol menthyl ether
Menthone glyceryl acetal
Menthone glyceryl ketal
Menthoxy-1,2-propandiol
Menthoxy-2-methyl-1,2-propanediol
Menthyl acetate
Menthyl ethylene glycol carbonate
Menthyl formiate
Menthyl glutamate
Menthyl glycerol carbonate
Menthyl hydroxy isobutyrate
Menthyl isobutyrate
Menthyl lactate
Menthyl malonate
Menthyl methyl ether
Menthyl N-ethyl oxamate
Menthyl propylene glycol carbonate
Menthyl pyroglutamate
Menthyl-(2-methoxy)acetate
Menthyl-(2-methoxyethoxy)acetate
Menthyl succinate
O-Menthyl succinic acid ester amide
O-Menthyl succinic acid ester-NN-(dimethyl)amide

Particularly preferred is menthyl acetate.

### Superabsorbers

Superabsorbent polymers (SAP) also called slush powder (component b) can absorb and retain extremely large amounts of a liquid relative to its own mass. Water-absorbing polymers, which are classified as hydrogels when mixed, absorb aqueous solutions through hydrogen bonding with water molecules. A SAP's ability to absorb water depends on the ionic concentration of the aqueous solution. In deionized and distilled water, a SAP may absorb 300 times its weight (from 30 to 60 times its own volume) and can become up to 99.9% liquid, but when put into a 0.9% saline solution, the absorbency drops to approximately 50 times its weight. The presence of valence cations in the solution impedes the polymer's ability to bond with the water molecule.

The total absorbency and swelling capacity are controlled by the type and degree of cross-linkers used to make the gel. Low-density cross-linked SAPs generally have a higher absorbent capacity and swell to a larger degree. These types of SAPs also have a softer and stickier gel formation. High cross-link density polymers exhibit lower absorbent capacity and swell, but the gel strength is firmer and can maintain particle shape even under modest pressure.

In the context of the present invention the preferred SAP are selected from the group consisting of polyacrylates, polyacrylamides, polyvinyl pyrrolidones, amylopectin, gelatin, cellulose and mixtures thereof. Superabsorbent polymers are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile to name a few. The latter is one of the oldest SAP forms created.

Today superabsorbent polymers are made using one of three primary methods: gel polymerization, suspension polymerization or solution polymerization. Each of the processes has their respective advantages but all yield a consistent quality of product.

Gel polymerization. A mixture of acrylic acid, water, cross-linking agents and UV initiator chemicals are blended and placed either on a moving belt or in large tubs. The liquid mixture then goes into a “reactor” which is a long chamber with a series of strong UV lights. The UV radiation drives the polymerization and cross-linking reactions. The resulting “logs” are sticky gels containing 60-70% water. The logs are shredded or ground and placed in various sorts of driers. Additional cross-linking agent may be sprayed on the particles' surface; this “surface cross-linking” increases the product's ability to swell under pressure—a property measured as Absorbency Under Load (AUL) or Absorbency Against Pressure (AAP). The dried polymer particles are then screened for proper particle size distribution and packaging. The gel polymerization (GP) method is currently the most popular method for making the sodium polyacrylate superabsorbent polymers now used in baby diapers and other disposable hygienic articles.

Solution polymerization. Solution polymers offer the absorbency of a granular polymer supplied in solution form. Solutions can be diluted with water prior to application, and can coat most substrates or used to saturate them. After drying at a specific temperature for a specific time, the result is a coated substrate with superabsorbency. For example, this chemistry can be applied directly onto wires and cables, though it is especially optimized for use on components such as rolled goods or sheeted substrates. Solution-based polymerization is commonly used today for SAP manufacture of co-polymers, particularly those with the toxic acrylamide monomer. This process is efficient and generally has a lower capital cost base. The solution process uses a water-based monomer solution to produce a mass of reactant polymerized gel. The polymerizations own exothermic reaction energy is used to drive much of the process, helping reduce manufacturing cost. The reactant polymer gel is then chopped, dried and ground to its final granule size. Any treatments to enhance performance characteristics of the SAP are usually accomplished after the final granule size is created.

Suspension polymerization. The suspension process is practiced by only a few companies because it requires a higher degree of production control and product engineering during the polymerization step. This process suspends the water-based reactant in a hydrocarbon-based solvent. The net result is that the suspension polymerization creates the primary polymer particle in the reactor rather than mechanically in post-reaction stages. Performance enhancements can also be made during, or just after, the reaction stage.

### Compositions

The composition according to the present invention may comprise compounds (a) and (b) in ratios by weight of from about 10:90 to about 5:95 and preferably of from about 0.1:99.9 to about 1:99.

The invention also encompasses a method for producing said compositions comprising or consisting of the following steps:
(a) providing an aqueous solution of at least one menthol derivative;
(b) providing at least one water-absorbing polymer and/or hydrogel; and
(c) bringing said at least one water-absorbing polymer and/or hydrogel into contact with said aqueous solution of at least one menthol derivative.

### Industrial Application

Another object of the present invention refers to a sanitary product comprising the composition. Preferably said product represents a diaper, particularly a baby diaper, a panty liner or a sanitary napkin.

The present invention also refers to the use of the compositions for fighting, masking and/or neutralizing malodors, particular female body odors.

### EXAMPLES

### Examples 1 to 3—Comparative examples C1 to C7

### Evaluation of Malodor Neutralization

The object of the following experiments has been the evaluation of performance against feminine hygiene malodor out of sniffer boxes. For this purpose 12 expert panelists evaluated the samples from sniffer boxes. The malodor was placed on different cotton balls and put into an aroma-tight air bag with a volume of 71 together with the raw materials placed in a petri dish. Perfume- and malodor intensities were evaluated on a continuous line scale with ten labels ranging from “no odor” (0) to “strongest imaginable” (10) odor with “moderate” (5) being the unperfumed malodor reference sample. Note that panelists only see the verbal anchors not the numbers (0-10) while rating. Results are arithmetic means of the individual panelist's ratings. Subsequently, the data obtained from the olfactory tests were evaluated statistically by a Friedmann-Test for overall difference between the samples followed by a pairwise comparison to identify significant differences among individual samples. The results are shown in Table 1:

TABLE 1
Feminine hygiene (to each sample 1 g water was added)

| Ex. | Sample | Perfume | Malodor |
|---|---|---|---|
| 0 | MO reference | 0.6 | 4.7 |
| C1 | MO + SAP | 0.1 | 5.4 |
| c2 | MO + menthyl acetate | 1.9 | 4.4 |
| C3 | MO + menthyl lactate | 1.9 | 4.3 |
| C4 | MO + menthone glycerin acetal | 1.9 | 4.4 |
| C5 | SAP + menthyl acetate | 3.7 | 0.7 |
| C6 | SAP + menthyl lactate | 3.7 | 0.7 |
| C7 | SAP + menthone glycerin acetal | 3.7 | 0.7 |
| 1 | MO + SAP + menthyl acetate | 3.6 | 3.5 |
| 2 | MO + SAP + menthyl lactate | 3.7 | 3.6 |
| 3 | MO + SAP + menthone glycerin acetal | 3.7 | 3.5 |

MO = Artificial malodor model for feminine hygiene
SAP = poly(methyl methacrylate-co-butyl methacrylate)

The results clearly indicate that adding one of the physiological cooling agents to said superabsorber malodor is significantly reduced. In particular, with regard to the samples containing “MO+superabsorber (+1 g water)+cooling agent” shows a significantly lower malodor intensity than “MO+superabsorber (+1 g water)” and “MO+cooling agent (+1 g water)”.

1. A composition for fighting malodors, comprising
(a) at least one physiological cooling agent, and
(b) at least one water-absorbing polymer and/or hydrogel.

2. The composition of claim 1, wherein said malodors are selected from the group consisting of female body odors, urine, ammonia, protein degradation products, and mixtures thereof.

3. The composition of claim 1, wherein said at least one physiological cooling agent is a menthol compound is selected from the group consisting of:
Menthol;
Menthol glyceryl acetal;
Menthol glyceryl ketal;
Menthol menthyl ether;
Menthone glyceryl acetal;
Menthone glyceryl ketal;
Menthoxy-1,2-propandiol;
Menthoxy-2-methyl-1,2-propanediol;
Menthyl acetate;
Menthyl ethylene glycol carbonate;
Menthyl formiate;
Menthyl glutamate;
Menthyl glycerol carbonate;
Menthyl hydroxy isobutyrate;
Menthyl isobutyrate;
Menthyl lactate;
Menthyl malonate;
Menthyl methyl ether;
Menthyl N-ethyl oxamate;
Menthyl propylene glycol carbonate;
Menthyl pyroglutamate;
Menthyl-(2-methoxy)acetate;
Menthyl-(2-methoxyethoxy)acetate;
Menthyl succinate;
O-Menthyl succinic acid ester amide;
O-Menthyl succinic acid ester-NN-(dimethyl)amide;
Menthane carboxylic acid-N-(4-cyanophenyl)amide;
Menthane carboxylic acid-N-(4-cyanomethylphenyl)amide;
Menthane carboxylic acid-N-ethylamide (WS-3);
(WS-4);
N^{α}-(menthane carbonyl) glycine ethylester (WS-5);
(1R,2S,5R)—N-(4-Methoxyphenyl)-5-methyl-2-(1-isopropyl)cyclohexane-carboxamide (WS-12);
(WS-14);
2,3-dimethyl-2-(2-propyl)-butyric acid-N-methylamide (WS23);
Isopulegol acetate;
p-Menthane-3,8-diol;
Cubebol;
3-Methyl-2(1-pyrrolidinyl)-2-cyclopentene-1-one);
Tetrahydropyrimidine-2-one;
N-(2-(pyridin-2-yl)ethyl)-3-p-menthanecarboxamide;
[(1R,2S, 5R)-2-isopropyl-5-methyl-cyclohexyl]2-(ethylamino)-2-oxo-acetate; and mixtures thereof.

4. The composition of claim 3, wherein said menthol compound is selected from the group consisting of:
Menthol glyceryl acetal;
Menthol glyceryl ketal;
Menthol menthyl ether;
Menthone glyceryl acetal;
Menthone glyceryl ketal;
Menthoxy-1,2-propandiol;
Menthoxy-2-methyl-1,2-propanediol;
Menthyl acetate;
Menthyl ethylene glycol carbonate;
Menthyl formiate;
Menthyl glutamate;
Menthyl glycerol carbonate;
Menthyl hydroxy isobutyrate;
Menthyl isobutyrate;
Menthyl lactate;
Menthyl malonate;
Menthyl methyl ether;
Menthyl N-ethyl oxamate;
Menthyl propylene glycol carbonate;
Menthyl pyroglutamate;
Menthyl-(2-methoxy)acetate;
Menthyl-(2-methoxyethoxy)acetate;
Menthyl succinate;
O-Menthyl succinic acid ester amide;
O-Menthyl succinic acid ester-NN-(dimethyl)amide; and mixtures thereof.

5. The composition of claim 4, wherein said menthol compound is menthyl acetate.

6. The composition of claim 1, wherein said at least one water-absorbing polymer is selected from the group consisting of polyacrylates, polyacrylamides, polyvinyl pyrrolidones, amylopectin, gelatin, cellulose, and mixtures thereof.

7. The composition of claim 1, wherein said at least one water-absorbing polymer is selected from the group consisting of polyacrylamide copolymers, ethylene maleic anhydride copolymers, cross-linked carboxymethyl celluloses, polyvinyl alcohol copolymers, cross-linked polyethylene oxides, starch grafted copolymers of polyacrylonitrile, and mixtures thereof.

8. The composition of claim 1 comprising compounds (a) and (b) in ratios by weight of from about 10:90 to about 5:95.

9. The composition of claim 1 comprising compounds (a) and (b) in ratios by weight of from about 0.1:99.9 to about 1:99.

10. A method for producing the composition of claim 1, comprising the following steps:
(a) providing an aqueous solution of at least one menthol derivative;
(b) providing at least one water-absorbing polymer and/or hydrogel; and
(c) bringing said at least one water-absorbing polymer and/or hydrogel into contact with said aqueous solution of at least one menthol derivative.

11. A sanitary product comprising the composition of claim 1.

12. The product of claim 12, which is a diaper, particularly a baby diaper.

13. The product of claim 12, which is a panty liner.

14. The product of claim 12, which is a sanitary napkin.

15. The method of claim 10, comprising the additional step of
(d) fighting, masking and/or neutralizing malodors, particular female body odors, by applying said thus-produced composition.
